(19) United States
(12) Patent Application Publication
(10) Pub. No.: US 2013/0079299 A1
(43) Pub. Date: Mar. 28, 2013

(54) Arthritis Therapeutic Agent

(71) Applicants: Hyung-Rae Cho, Haeundae-gu (KR); Kun-Ju Yang, Gangseo-gu (KR); Joo-Wan Kim, Masan-si (KR); Chae-Hun Ra, Saha-gu (KR); Min-Kyoeng Kim, Haeundae-gu (KR)

(72) Inventors: Hyung-Rae Cho, Haeundae-gu (KR); Kun-Ju Yang, Gangseo-gu (KR); Joo-Wan Kim, Masan-si (KR); Chae-Hun Ra, Saha-gu (KR); Min-Kyoeng Kim, Haeundae-gu (KR)

(73) Assignee: GLUCAN CORPORATION, Busan (KR)

(21) Appl. No.: 13/697,510

(22) PCT Filed: May 12, 2010
(86) PCT No.: PCT/KR2010/003008

§ 371 Date: Nov. 30, 2012

(51) Int. Cl. A61K 31/716

(52) U.S. Cl. CPC A61K 31/716
USPC 514/54; 536/123.12

(57) ABSTRACT

Disclosed is a composition for preventing, treating, or alleviating arthritis, and particularly, a composition effective for preventing, treating, or alleviating arthritis comprising β-1,3-1,6-branched D-glucan. The β-1,3-1,6-branched D-glucan of the composition is characterized in that glucoses are linked via a beta-1,3 linkage between two glucose molecules and a gluclose is branched via a linkage between 1 and 6 positions of two glucose molecules in every 1-20 of the beta-1,3 linked glucoses, and the branched glucose is bound with an organic acid. Further, an arthritis therapeutic agent and a health supplement food including the composition, and a method of preventing, treating, or alleviating arthritis including administering a pharmaceutically effective amount of the composition, are disclosed.

### TECHNICAL FIELD

The present invention relates to a composition for preventing, treating, or alleviating arthritis; a method of preventing, treating, or alleviating arthritis including administering the composition; and a health supplement agent effective for preventing, treating, or alleviating arthritis including the composition. More particularly, the composition comprises β-1,3-1,6-branched D-glucan, wherein the β-1,3-1,6-branched D-glucan is characterized in that glucoses are linked via beta-1,3 linkage between two adjacent glucose molecules to form a linear chain, and a glucose is branched from the chain at every 1 to 20 of the glucoses in the chain, in which the branched glucose is linked to the chain via a linkage between 1 and 6 positions of two glucose molecules, and wherein the branched glucose is bound with an organic acid.

### BACKGROUND ART

The human body includes about 200 joints. A joint is a junction connecting bone to bone. The joint includes cartilage, an articular capsule, a synovial membrane, ligaments, tendons, muscles, or the like, and absorbs impact generated by the motion for smooth movement between bones.

Inflammatory diseases of joints may be broadly divided into chronic rheumatoid arthritis that is understood to be caused by auto-immune disorders, infectious arthritis caused by bacterial infection, degenerative arthritis in which articular cartilage or bone is degenerated or destroyed due to various causes, and crystal arthritis in which soluble metabolism products are deposited in connective tissue around the joint as crystals due to a degenerative change.

Degenerative arthritis, that is, osteoarthritis, is a disease caused by degeneration of chondrocytes of a joint due to aging or the like, thereby suppressing the synthesis of type II collagen and proteoglycan or the like, which are joint substrate materials, in the chondrocytes, and simultaneously producing an inflammatory cytokine such as interleukin-1β and tumor necrosis factor-α or the like, thereby increasing the synthesis and the activity of matrix metalloproteinase for decomposing the joint substrate in the joint cells, resulting in destruction of the joint tissue.

In addition, nitrogen monoxide is generated by the inflammatory cytokine, and then self-amplifying cytokine is produced due to the produced nitrogen monoxide so as to induce further more synthesis of MMP and to accelerate the decomposition of the joint substrate, so that the arthritis is worsened. Simultaneously, the inflammatory cytokine accelerates the production of prostagladine E2, which is a lipid metabolism product, causing the inflammatory response in arthritis.

Rheumatoid arthritis is a chronic, systemic inflammatory disease, causing symmetric polyarthritis and thereby damage and deformation of joint occur. When rheumatoid arthritis is not treated, it leads to a disorder of joint functions, and may negatively affect the daily lives of a sufferer if it persists. In Korea, it is estimated that about 1% of the population is affected by rheumatoid arthritis, and three times as many women are affected by than men, and its onset is most frequent between the ages of 20 and 40.

The key causative factors of rheumatoid arthritis have been gradually discovered, which are thought to be genetic factors, hormone disorders, or the like. Auto-immunity occurs because of the causative factors. Auto-immunity is a phenomenon in which chronic inflammation is frequently and continuously induced in different body parts due to damage to the immune regulation system.

Drugs for treating arthritis may be broadly divided according to the main action metabolisms, as follows: decreasing inflammation, delaying the progression of the disease, and decreasing uric acid concentration. The many therapeutic agents of nervous arthritis play a role of decreasing inflammation. Inflammation is a pathological process that induces pain, edema, fever, stroke, or spasticity, and therapeutic agents for relieving inflammation include non-steroidal anti-inflammatory drugs such as aspirin and steroidal anti-inflammatory drugs such as cortisone.

The non-steroidal anti-inflammatory drugs relieve joint pain and alleviate inflammation, but they may cause gastrointestinal disorders and abdominal pain. Thus, they are contraindicated for people having a medical history of gastrointestinal bleeding or active peptic ulcers. The steroidal anti-inflammatory drugs have serious side effects such as weight gain and high blood pressure, so they are rarely used for degenerative nervous arthritis.

Particularly, steroidal anti-inflammatory drugs are not therapeutic agents to treat the cause of disease but only temporarily reduce pain, so they may be used in an excessive amount causing destruction and damage to the joint, so careful use thereof is required.

Accordingly, the conventional treatment for joint damage such as arthritis has limited effectiveness, and accompanies clear toxic side effects. Also, it may be not effective in the long term, so a novel treatment or therapeutic agent is required for overcoming the drawbacks of the conventional treatment.

### DISCLOSURE

### Technical Solution

The present invention provides a composition for preventing, treating, or alleviating arthritis, including β-1,3-1,6-branched D-glucan, an arthritis therapeutic agent and a health supplement food including the composition, and a method of treating arthritis including administering the composition. The arthritis may be osteoarthritis or rheumatoid arthritis.

One embodiment of the invention provides a composition for preventing, treating, or alleviating arthritis comprising β-1,3-1,6-branched D-glucan.

Specifically, the β-1,3-1,6-branched D-glucan is characterized in that glucoses are linked via beta-1,3 linkage between two adjacent glucose molecules to form a linear chain, and a glucose is branched from the chain at every 1 to 20 of the glucoses in the chain, in which the branched glucose is linked to the chain via a linkage between 1 and 6 positions of two glucose molecules, and wherein the branched glucose is bound with an organic acid.

The organic acid bound to the branched glucose may be selected from the group consisting of lactic acid, oxalic acid, oxalacetic acid, fumaric acid, malic acid, succinic acid, acetic acid, butyric acid, palmitic acid, tartaric acid, ascorbic acid, uric acid, sulfonic acid, sulfinic acid, phenol, tartaric acid, formic acid, citric acid, isocitric acid, alpha-ketoglutaric acid, succinic acid, hexane, PGAL, DPGA, and PGA.

The composition may be administered at 21.25 mg to 85 mg per 1 kg of body weight.

The arthritis may be osteoarthritis (degenerative arthritis) or rheumatoid arthritis.

Another embodiment of the invention provides an osteoarthritis therapeutic agent including the composition.

Another embodiment of the invention provides a therapeutic agent for rheumatoid arthritis comprising the composition.

Another embodiment of the invention provides a method of treating, preventing, or alleviating arthritis comprising administering the composition. The arthritis may be osteoarthritis or rheumatoid arthritis. Preferably, the composition is characterized by administering at 21.25 mg to 85 mg per 1 kg of body weight.

Another embodiment of the invention provides a health supplement agent effective for preventing, treating, and alleviating arthritis comprising the composition. The health supplement agent may be a health supplement food.

### Advantageous Effects

The composition and the arthritis therapeutic agent including the composition according to the present invention may provide a novel therapeutic agent having less side reaction and treating the causes of arthritis to overcome the drawbacks of the conventional therapeutic agent. dr

### DESCRIPTION OF THE DRAWINGS

FIG. 1 is a graph showing weight change by day of animal models of a non-treatment group (OA control) after causing osteoarthritis, a group administered diclofenac sodium, and groups administered 85, 42.5, and 21.25 mg/kg of Polycan, respectively.

FIG. 2 is a graph showing the thickness change of knee joint by day of the animal models of the non-treatment group (OA control) after causing osteoarthritis, the group administered diclofenac sodium, and the groups administered 85, 42.5, and 21.25 mg/kg of Polycan, respectively.

FIG. 3 is a graph showing the knee joint thickness and the maximum stretching angle by day of the animal models of a sham control group administered sterilized distilled water without causing osteoarthritis, the group administered diclofenac sodium after causing osteoarthritis, and the groups administered 85, 42.5, and 21.25 mg/kg of Polycan, respectively.

FIG. 4 is a graph showing the Mankin score of animal models of the group administered diclofenac sodium after causing osteoarthritis, and the groups administered 85, 42.5, and 21.25 mg/kg of Polycan, respectively.

FIG. 5 is a graph showing the articular cartilage thickness after amputating the joint portion of animal models of the group administered diclofenac sodium after causing osteoarthritis, and the groups administered 85, 42.5, and 21.25 mg/kg of Polycan, respectively.

FIG. 6 is a graph showing the cartilage cell number of animal models of the group administered diclofenac sodium after causing osteoarthritis, and the groups administered 85, 42.5, and 21.25 mg/kg of Polycan, respectively.

FIG. 7 shows the thymus cortex thickness of the sham control group (a, b) administered sterilized distilled water without causing osteoarthritis, the rheumatoid arthritis control (RA control) group (c, d), the group administered diclofenac sodium (e, f) after causing rheumatoid arthritis, and the groups administered 85 (k, l), 42.5 (i, j), and 21.25 (g, h) mg/kg of Polycan, respectively. Herein, C denotes a cortex region and M denotes a marrow region.

FIG. 8 shows the splenic artery of the sham control group (a, b) administered sterilized distilled water without causing osteoarthritis, the rheumatoid arthritis control (RA control) group (c, d), the group administered diclofenac sodium (e, f) after causing rheumatoid arthritis, and the groups administered 85 (k, l), 42.5 (i, j), and 21.25 (g, h) mg/kg of Polycan, respectively. Herein, W denotes a white pulp region, R denotes a red pulp region, and A denotes a central arteriole.

FIG. 9 shows the surface of the femur knee joint of the sham control group (a) administrating sterilized distilled water without causing arthritis, the rheumatoid arthritis control (RA control) group (b), the group administered diclofenac sodium (c) after causing rheumatoid arthritis, and the groups administered 85 (f), 42.5 (e), and 21.25 (d) mg/kg of Polycan, respectively. Herein, S denotes a synovial cavity of the knee joint, and B denotes a fine bone.

FIG. 10 shows the surface of the tibia knee joint of the sham control group (a) administered sterilized distilled water without causing arthritis, the rheumatoid arthritis control (RA control) group (b), the group administered diclofenac sodium (c) after causing rheumatoid arthritis, and the groups administered 85 (f), 42.5 (e), and 21.25 (d) mg/kg of Polycan, respectively. Herein, S denotes a synovial cavity of the knee joint, and B denotes fine bones.

### MODE FOR INVENTION

Exemplary embodiments of this disclosure will hereinafter be described in detail referring to the drawings. However, these embodiments are only exemplary, and this disclosure is not limited thereto.

The composition for preventing, treating, or alleviating arthritis according to the present invention comprises β-1,3-1,6-branched D-glucan. In the β-1,3-1,6-branched D-glucan, glucoses are linked via beta-1,3 linkage between two adjacent glucose molecules to form a linear chain. The pharmaceutical composition according to the present invention is effective for preventing, treating, or alleviating arthritis, particularly, osteoarthritis and rheumatoid arthritis.

The glucose promotes cartilage formation by stimulating chondrocytes. In the β-1,3-1,6-branched D-glucan, a glucose is branched from the chain at every 1 to 20 of the glucoses in the chain, in which the branched glucose is linked to the chain via a linkage between 1 and 6 positions of two glucose molecules. Preferably, glucose is branched in every 5 glucoses that are linked via a beta-1,3 linkage. When the number of glucoses that are linked via a beta-1,3 linkage, from which a glucose is branched, is less than 1, the effect on promoting the cartilage formation may be deteriorated, while when the number is more than 20, the cartilage formation effects are deteriorated since the molecular weight is excessively increased to be absorbed in the body. Therefore, it is preferable to provide a glucose chain in which a glucose is branched via a linkage between 1 and 6 positions of two glucose molecules in every 1 to 20 glucoses that are linked via a beta-1,3 linkage between two glucose molecules. The glucose may be linked in linear, branched, or cyclic fashions.

In addition, the β-1,3-1,6-branched D-glucan may comprise a repeating unit of following Chemical Formula 1.

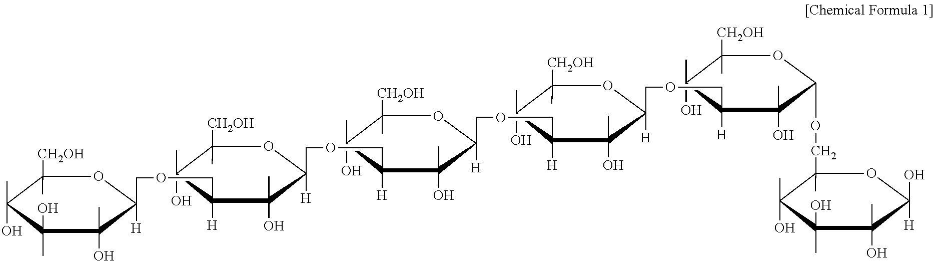

In above Chemical Formula 1, the side chain of the 1,6-branched glucose may be bound with an organic acid. Examples of the organic acid may include lactic acid, oxalic acid, oxalacetic acid, fumaric acid, malic acid, succinic acid, acetic acid, butyric acid, palmitic acid, tartaric acid, ascorbic acid, uric acid, sulfonic acid, sulfinic acid, phenol, tartaric acid, formic acid, citric acid, isocitric acid, alpha-ketoglutaric acid, succinic acid, hexane, PGAL, DPGA, and PGA or the like. Preferably, the organic acid is lactic acid. The organic acid promotes to adsorb calcium and activates chondrocytes.

On the other hand, the composition according to the present invention may be administered to a mammal such as a rat, a mouse, livestock, a human, and the like through various administration routes. It may be administered by all routes, for example, orally or rectally, or by intravenous, intramuscular, hypodermic, intrauterine, dural, or intracerebroventricular injection. The preferable administrative amount differs depending upon the status and the weight of the patient, the seriousness of disease, drug formation, and the administration route and period, so it may be appropriately selected by a person of ordinary skill in the art.

The composition is preferably administered in an amount of about 21.25 mg/kg to 85 mg/kg. When the composition is administered in an amount of less than 21.25 mg/kg, the arthritis may be insufficiently treated or prevented, and when it is more than 85 mg/kg, the effect may be deteriorated, so it is preferable to stay within the range.

The composition may be used as an osteoarthritis therapeutic agent and a rheumatoid arthritis therapeutic agent. Osteoarthritis causes cartilage loss and the joint stiffening. The pharmaceutical composition effectively suppresses the symptoms and also very effectively suppresses the joint loss of the tibia and femur.

Osteoarthritis is an inflammatory disease which causes edema around joints due to cartilage damage or the like, and causes a remarkable increase of joint thickness [Guo et al., 2006]. The composition remarkably enhances immunoreactive cells and also increases the cartilage in order to prevent inflammation.

In the case of the rheumatoid arthritis, the joint is seriously swollen due to a disorder of the auto-immune system, so the pharmaceutical composition may prevent, treat, or alleviate rheumatoid arthritis by controlling the immune system or the anti-inflammatory effects.

The composition may be formulated in an oral formulation such as powder, granules, a tablet, a capsule, a suspension, an emulsion, a syrup, an aerosol, or the like, or an external preparation, a suppository, or a sterilized injection solution.

The composition according to the present invention may further include glucosamine, chondroitin, hyaluronic acid, methylsulfonyl methane, and creatine or appropriate derivatives thereof, and/or a physiologically active component additive such as a formulation agent, a stabilizer, a filler, a flavoring agent, a dye, and a sweetener.

The carrier, excipient, and diluents which may be included in the composition may include lactose, dextrose, sucrose, sorbitol, mannitol, xylitol, erythritol, maltitol, starch, acacia rubber, alginate, gelatin, calcium phosphate, calcium silicate, cellulose, methyl cellulose, microcrystalline cellulose, polyvinyl pyrrolidone, water, methylhydroxy benzoate, propylhydroxy benzoate, talc, magnesium stearate, and mineral oil.

It may be formulated with generally used diluents or excipients such as a filler, an extending agent, a binder, a wetting agent, a disintegrating agent, a surfactant, or the like.

The solid formulation for the oral administration may include a tablet, a pill, a powder, granules, a capsule, or the like, and the solid formulation may further include at least one excipient. The excipient may include, for example, starch, calcium carbonate, sucrose, lactose, gelatin, or the like, and may be formulated by mixing with the solid formulation. Besides the simple excipients, it may include lubricants such as talc and magnesium stearate.

The liquid formation for the oral administration may include a suspension, a liquid solution, an emulsion, a syrup, or the like, and may further include simple diluents such as water, liquid paraffin, an excipient such as a wetting agent, a flavoring agent, a freshener, a preservative, or the like.

The formulation for the parenteral administration may include a sterilized aqueous solution, a non-aqueous solute, a suspension, an emulsion, a lyophilized formulation, and a suppository. The non-aqueous solute and the suspension may include propylene glycol, polyethylene glycol, vegetable oil such as olive oil, an injectionable ester such as ethylolate, or the like. The substrate for the suppository may include witepsol, macrogol, tween 61, cacao butter, laurinum, glycerogelatin, or the like.

The composition may be applied for a health supplement agent for preventing or alleviating arthritis as well as for the arthritis therapeutic agent as in the above.

The health supplement agent may be supplied as a food. The “health functional food” defined in the specification means a food manufactured and processed by using a raw material or a component having useful functions for the human body set in Law for Health Functional Foods (No. 6727), and the term “functional” means eating for obtaining the useable effects for the health care usage such as for controlling nutrients or physiological functions or the like in the human body structure and function.

Further, the health supplement agent according to the present invention may include various nutritional supplements, vitamins, minerals (electrolytes), a flavoring agent such as a synthesized flavoring agent, a natural flavoring agent, or the like, a colorant, an enhancer (cheese, chocolate, etc.), pectic acid and a salt thereof, alginic acid and a salt thereof, an organic acid, a protective colloidal thickener, a pH controlling agent, a stabilizer, an preservative, glycerin, alcohol, a carbonating agent for carbonated beverages, and the like.

The health supplement agent according to the present invention includes the essential components at the indicated ratios and other additional components without limitation. The additional components may include various flavoring agents, natural carbohydrates, or the like as in a general beverage. Examples of natural carbohydrates may include a sugar such as a monosaccharide such as glucose, fructose, or the like, a disaccharide such as maltose, sucrose, or the like, and a polysaccharide such as dextrin, cyclodextrin, or the like, and a sugar alcohol such as xylitol, sorbitol, erythritol ,or the like. The flavoring agent excepting the above-mentioned ones may include a natural flavoring agent (thaumatin, Stevia extract (e.g., Rebaodiocide A, glycyrrhizin, or the like) and a synthetic flavoring agent (saccharin, aspartame, or the like). The ratio of the natural carbohydrate generally ranges from about 1 to 20 g, preferably, from about 5 to 12 g, per 100 ml of the composition according to the present invention.

Hereinafter, the effects on osteoarthritis and rheumatoid arthritis are explained using Polycan™ [Glucan Corp. Ltd., Korean] which is one example including the composition according to the present invention with reference to the drawings. Hereinafter, the embodiments are illustrated in more detail with reference to examples. However, the followings are exemplary embodiments and are not limiting.

### Experimental Example 1

### Verifying Efficacy of ‘β-1,3-1,6-branched D-glucan’

### Experimental Example 1-1

### Test Preparation

Polycan™ (1.7brix) [Glucan Corp. Ltd., KOREA], one of commercially available β-1,3-1,6-branched D-glucan, was prepared, and diclofenac sodium [Sigma, USA] was prepared as a control drug of the Polycan™. Sprague-Dawley rats (male, 6 week-old, SLC., JAPAN) [ANNEX I-III] were prepared as laboratory animals.

### Experimental Example 1-2

### Separating Groups

Six experimental groups were formed, with 8 Sprague-Dawley Rats in each group. A sham control group included normal rats which were not caused to have osteoarthritis and were administered sterilized distilled water. In addition, a negative control group was an OA control group which was treated with nothing after causing osteoarthritis, and a positive control group was administered 2 mg/kg of diclofenac sodium after causing osteoarthritis.

The treated groups were administered Polycan in different concentrations of 85 mg/kg, 42.5 mg/kg, and 21.25 mg/kg after causing osteoarthritis.

### Experimental Example 1-3

### Administration

Each of 85, 42.5, and 21.25 mg/kg of Polycan was oral administered every day for 84 days. The medium for the administration was sterilized distilled water at 5 ml/kg. In addition, Polycan was diluted in a culture medium, and a concentration of 1 ml/kg was injected once into the articular capsule.

For the positive control, 2 mg/kg of the diclofenac sodium was transdermally administered at 1 ml/kg of normal saline as a medium every day for 84 days. The Polycan diluted in a culture medium was injected from one week after causing osteoarthritis, and the diclofenac sodium was also administered from one week after causing osteoarthritis.

### Experimental Example 1-4

### Causing Osteoarthritis

Each experimental rat was anesthetized with Zoletile 50 (Vir bac Lab., France), and the left articular capsule was exposed and underwent anterior cruciate ligament transaction and partial medial meniscectomy to cause osteoarthritis. For the sham surgery group, the articular capsule was excised to observe the medial meniscus inside the organ and closed without surgical removal.

### Experimental Example 1-5

### Observation

Weight change and knee joint thickness change were monitored and measured once a week for 84 days from administration, and on the final day, the maximum stretching angle of the knee joint and the knee joint thickness after exposing the capsule were measured under Safranin O dye, respectively. The Makin scores of the femur and tibia and the cartilage thickness were then histomorphometrically measured, and the articular cartilages of the femur and tibia were each evaluated for BrdU immunoreactivity.

### Experimental Example 2

### Analyzing Efficacy of ‘β-1,3-1,6-branched D-glucan’ (Polycan™) on Osteoarthritis

### Experimental Example 2-1

### Weight Change

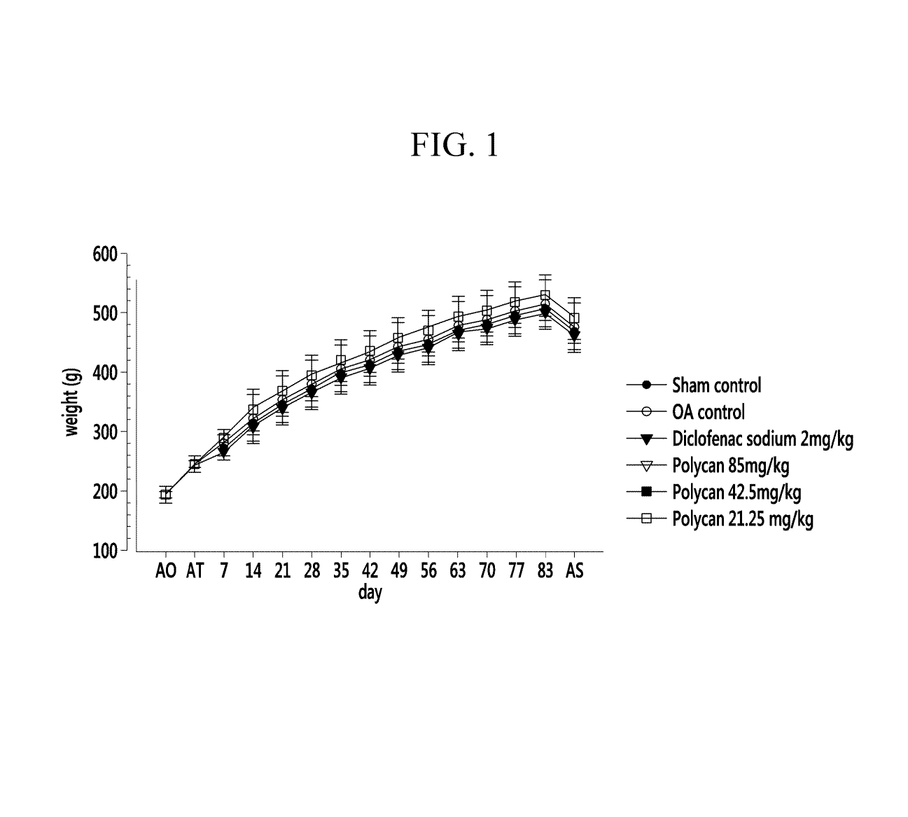

As shown in FIG. 1, significant weight change and weight gain were not observed in the sham control group or the negative control group (OA control) from the results of Experimental Example 1. The weight of the OA control group through 84 days was changed by −0.82% compared to that of the sham control group, and the groups administered 2 mg/kg of diclofenac sodium and 85, 42.5 and 21.25 mg/kg of Polycan changed by −2.98, 1.76, 7.76, and 8.88%, respectively, compared to the OA control group.

### Experimental Example 2-2

### Knee Joint Thickness Change

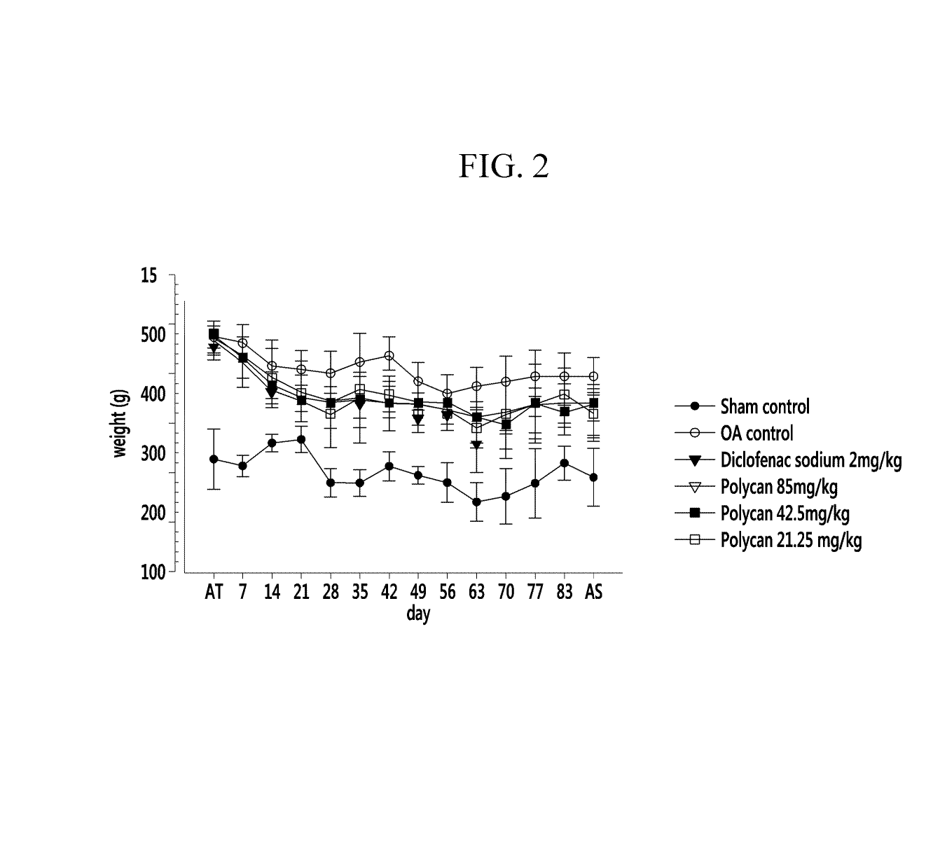

Referring to FIG. 2, in the osteoarthritis control group (OA control), it was observed that the induced thickness of the knee joint was significantly increased (p<0.01) compared to that of the sham control group from the administration day. On the other hand, it is seen that, in each of the three-dose Polycan and diclofenac sodium administration groups, the thickness of the knee joint was significantly decreased (p<0.01 or p<0.05) compared to that of osteoarthritis-caused control group from 21 days after administration. In the final autopsy, the thickness of the knee joint of the OA control group was changed by 18.66% compared to that of the sham control group, and the groups administered 2 mg/kg of diclofenac sodium, and 85, 42.5, and 21.25 mg/kg of Polycan changed by −5.79, −5.61, −4.28, and −5.74%, respectively, compared to the OA control group.

### Experimental Example 2-3

### Knee Joint Thickness Change after Exposing Capsule

In all OA groups, the joint thickness after exposing the knee capsule was significantly increased (p<0.01) compared to that of the sham control group, and all groups administered diclofenac sodium and Polycan showed a similar thickness to the OA-induced control group. The thickness of the knee joint after exposing the joint capsule of the OA control group was changed by 15.40% compared to that of the sham control group, and the groups administered 2 mg/kg of diclofenac sodium and 85, 42.5, and 21.25 mg/kg of Polycan changed by −0.99, −2.77, −1.00, and −2.17%, respectively, compared to the OA control group.

### Experimental Example 2-4

### Maximum Stretching Angle of Knee Joint

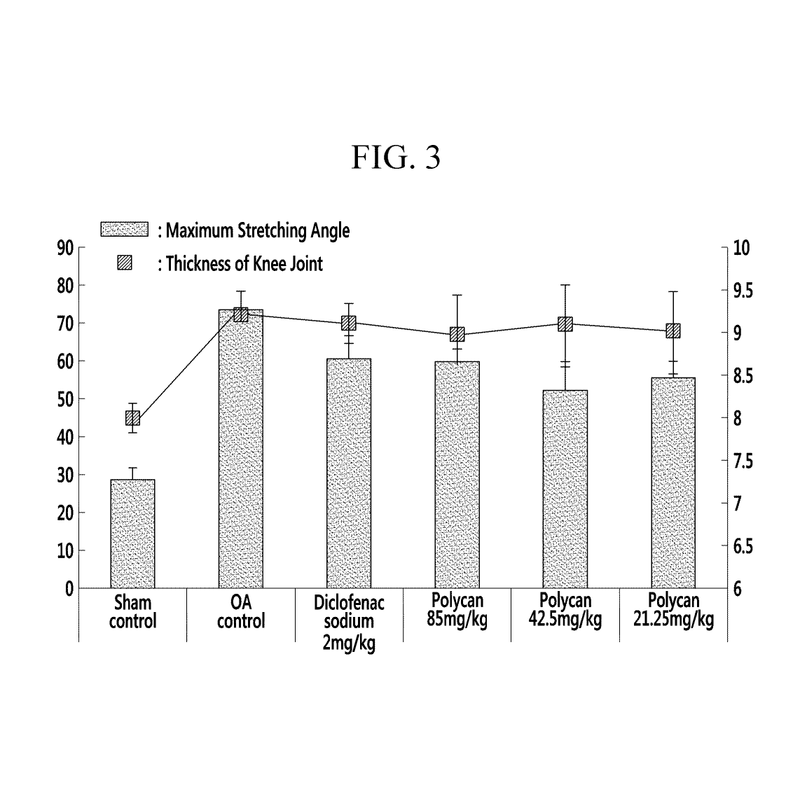

Referring to FIG. 3, it is seen that the maximum stretching angle of the OA control group was significantly increased (p<0.01) compared to that of the sham control group, and the groups administered Polycan and diclofenac sodium significantly decreased (p<0.01) the maximum stretching angle compared to the OA control group. The maximum stretching angle of knee joint of the OA group changed by 159.39% compared to that of the sham control group, and the group administered 2 mg/kg of diclofenac sodium and 85, 42.5, and 21.25 mg/kg of Polycan changed by −18.18, −18.52, −28.96, and −24.07%, respectively, compared to the OA control group.

### Experimental Example 2-5

### Mankin Score

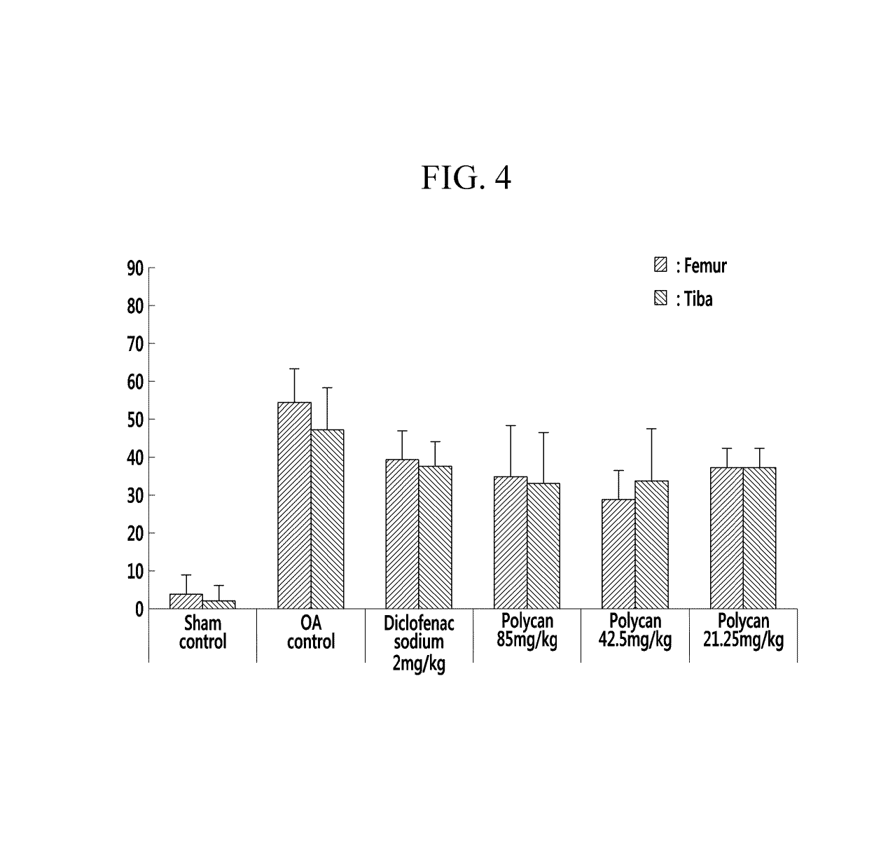

The Mankin score refers to a value showing the seriousness of arthritis and evaluating all of stretching angle, pain, fever, thickness, or the like. Smaller scores approach the normal. Referring to FIG. 4, it is seen that Mankin scores of the tibia and femur articular cartilage of the OA control group were significantly increased (p<0.01) compared to that of the sham control group, but all groups administered Polycan and diclofenac sodium remarkably decreased in Mankin scores of the femur and tibia compared to the OA control group.

The Mankin score of the femur of the OA control was changed by 1216.67% compared to that of the sham control group, and the group administered 2 mg/kg of diclofenac sodium and 85, 42.5, and 21.25 mg/kg of Polycan changed by −27.85, −36.71, −48.10, and −31.65%, respectively, compared to the OA control group.

The Mankin score of the tibia of the OA control was changed by 2166.67compared to that of the sham control group, and the group administered 2 mg/kg of diclofenac sodium and 85, 42.5, and 21.25 mg/kg of Polycan changed by −20.59, −29.41, −27.94, and −20.59%, respectively, compared to the OA control group.

### Experimental Example 2-6

### Change of Cartilage Thickness

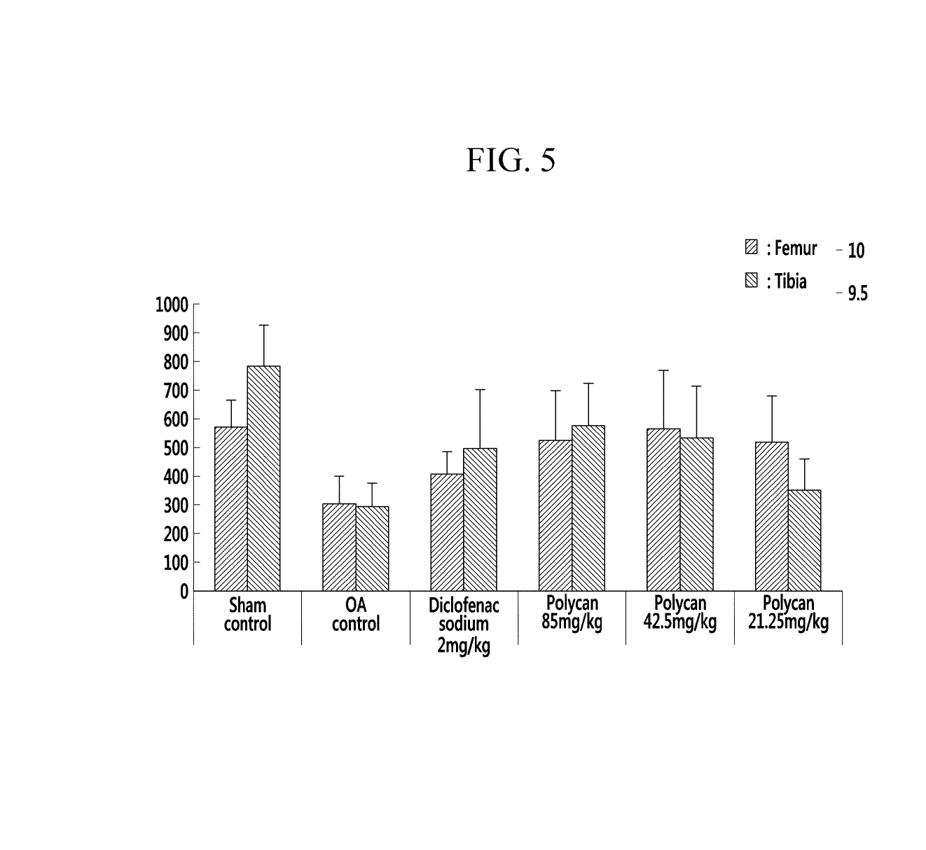

Referring to FIG. 5, it is seen that the articular cartilage thickness of the tibia and femur of the OA control were significantly decreased (p<0.01) compared to that of the sham control group, but all group administered Polycan and diclofenac sodium had significantly increased (p<0.01) thickness of the articular cartilage of the tibia and femur compared to the OA control group excepting one group administered 21.25 mg/kg of Polycan.

On the other hand, it is seen that, in the group administered 21.25 mg/kg of Polycan, the femur cartilage thickness was significantly (p<0.01) increased, and the tibia cartilage thickness was also increased.

The articular cartilage thickness of the femur of the OA control group was changed by −46.78% compared to that of the sham control group, and the groups administered 2 mg/kg of diclofenac sodium and 85, 42.5, and 21.25 mg/kg of Polycan changed the by 35.11, 71.84, 86.87, and 69.38%, respectively, compared to the OA control group.

The articular cartilage thickness of the femur of the OA control group was changed by −61.99% compared to that of the sham control group, and the groups administered 2 mg/kg of diclofenac sodium and 85, 42.5, and 21.25 mg/kg of Polycan changed by 68.81, 96.23, 79.51, and 18.74%, respectively, compared to the OA control group.

### Experimental Example 2-7

### Change of BrdU Immunoreactivity

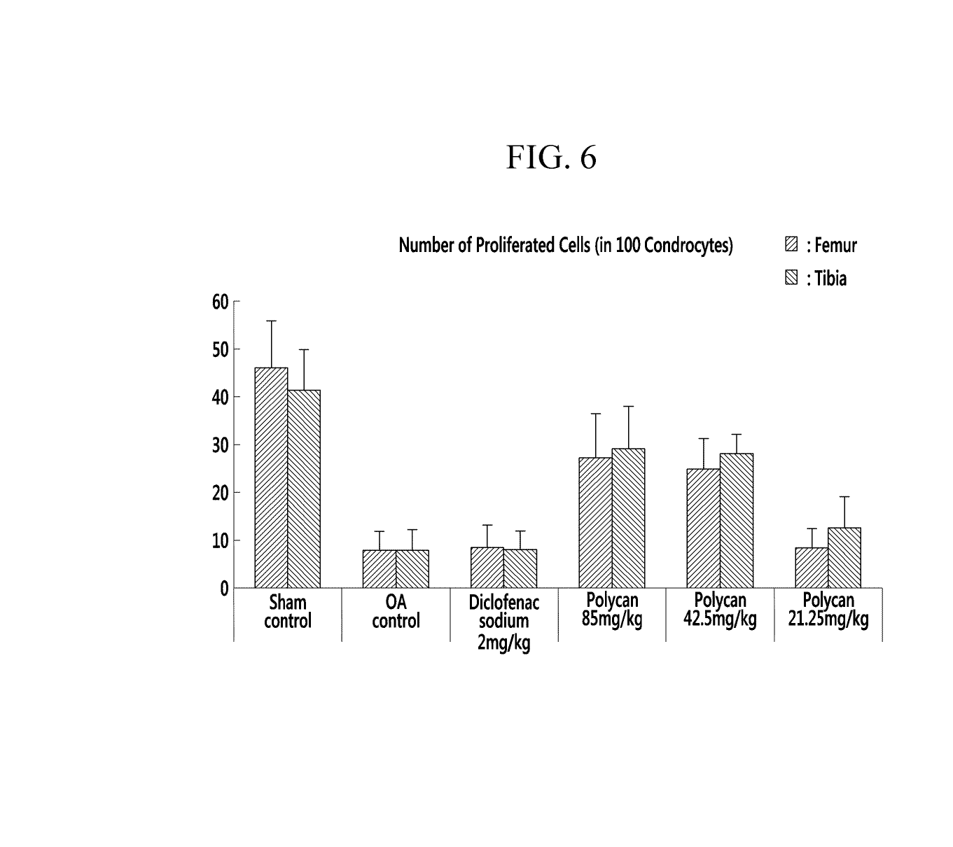

Referring to FIG. 6, it is seen that the BrdU immunoreactive cell number of the tibia and femur articular cartilage of the OA control group was significantly decreased (p<0.01) compared to that of the sham control group, but groups administered 85 and 42.5 mg/kg of Polycan had a significantly increased (p<0.01) number of chondrocytes having BrdU immunoreactivity in both tibia and femur articular cartilages compared to the OA control group.

On the other hand, it is seen that the group administered diclofenac sodium showed a similar BrdU immunoreactive cell number to the OA control group in both the tibia and femur, and the group administered 21.25 mg/kg of Polycan had a remarkably increased number of BrdU immunoreactive cells compared to the OA control group in the tibia joint cartilage and showed a similar cell number to the OA control in the femur joint cartilage.

In the femur articular cartilage of the OA control group, the BrdU immunoreactive cell number was changed by −82.69% compared to that of the sham control group, and the groups administered 2 mg/kg of diclofenac sodium, and 85, 42.5, and 21.25 mg/kg of Polycan changed by 9.52, 239.68, 207.94, and 6.35%, respectively, compared to the OA control group.

In the tibia articular cartilage of the OA control group, the BrdU immunoreactive cell number was changed by −80.43% compared to that of the sham control group, and the groups administered 2 mg/kg of diclofenac sodium, and 85, 42.5, and 21.25 mg/kg of Polycan changed by 1.56, 259.38, 245.31, and 57.81%, respectively, compared to the OA control group.

### Experimental Example 3

### Verifying Rheumatoid Therapeutic Efficacy of ‘β-1,3-1,6-branched D-glucan’ (Polycan™)

### Experimental Example 3-1

### Tissue Preparation

Bone tissue was amputated from around the thymus and spleen and fixed in 10% neutral formalin, and was then dehydrated and paraffin embedded to provide a 3-4 μm longitudinal section, then dyed with Hematoxylineosin and observed under an optical microscope.

In addition, the knee joint of both the rear legs were removed from the around connective tissue and fixed in 10% neutral formalin, and then it was decalcificated using a decalcification solution containing 24.4% of formic acid and 0.5 N of sodium hydroxide for 5 days. Then, it was dehydrated and subjected to the paraffin embedding according to the general method, and then was formed as 3-4 μm longitudinal section. Then the section was dyed with Hematoxylineosin and observed under the optical microscope.

### Experimental Example 3-2

### Histomorphometry

In order to evaluate the efficacy of Polycan on the immune disorder of the rheumatoid arthritis (RA) DBA mouse caused by collagen, the thickness of the entire thymus and cortex, the diameter of the entire spleen and white medulla, and the number of white medulla per unit area of 1 mm² were respectively measured by an automated image analyzer (DMI-300 Image Processing; DMI, Korea) in the view of a 40×-magnification microscope, and the thickness of articular cartilage of the femur and tibia of the knee joint, the proteoglycan loss, and erosion were evaluated by an automated image analyzer (DMI-300 Image Processing; DMI, Korea) in the view of a 100×-magnification microscope according to the known method [Dudler et al., 2000; van Holten et al., 20 04; Kim et al., 2007].

The proteoglycan loss was evaluated into 3 levels for stainability of Safranin O dye specialized for proteoglycans according to the known method [Williams et al., 1992; Dudler et al., 2000; van Holten et al., 2004]. In other words, 0 indicates no proteoglycan loss, and 3 indicates no stainability because of complete deflection.

The damage of the joint cartilage was evaluated according to the known methods [Williams et al., 1992; van Holten et al., 2004; Kim et al., 2007] as 4 levels from level 0 (seen as no erosion) to level 4 (even bone tissue was damaged due to serious erosion).

### Experimental Example 3-3

### Animal Model

The RA(Rhematoid Arthritis)-induced mouse model using collagen is one of the animal models that are widely used for evaluating the efficacy on RA for various materials [Liu et al., 2008; Miyake et al., 2008; Pa nayi et al., 2008], and RA was caused by the auto-immunity. Accordingly, as a typical immune increase such as a lymphocyte increase of the thymus and spleen is known during collagen-induced RA [Agata et al., 2000; Chen and Wei, 2003; Zhang et al., 2004], the thymus and spleen of RA-induced mouse using collagen were observed to analyze the efficacy of the present invention.

### Experimental Example 3-4

### Thymus and Spleen Anatomy of Animal Model

From the results of the experimental examples, it is seen that only the groups administered diclofenac sodium and Polycan at 85 mg/kg insignificantly increased the thickness of thymus cortex, but a significant change was not seen in all RA-induced groups compared to that of the sham control group.

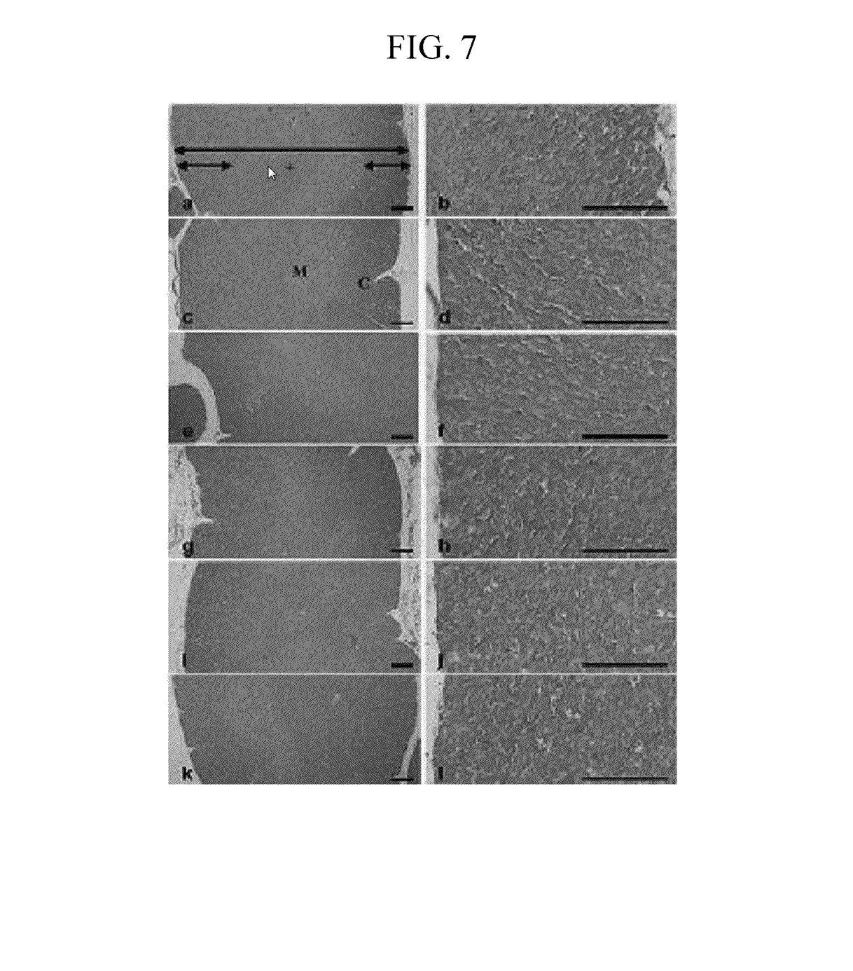

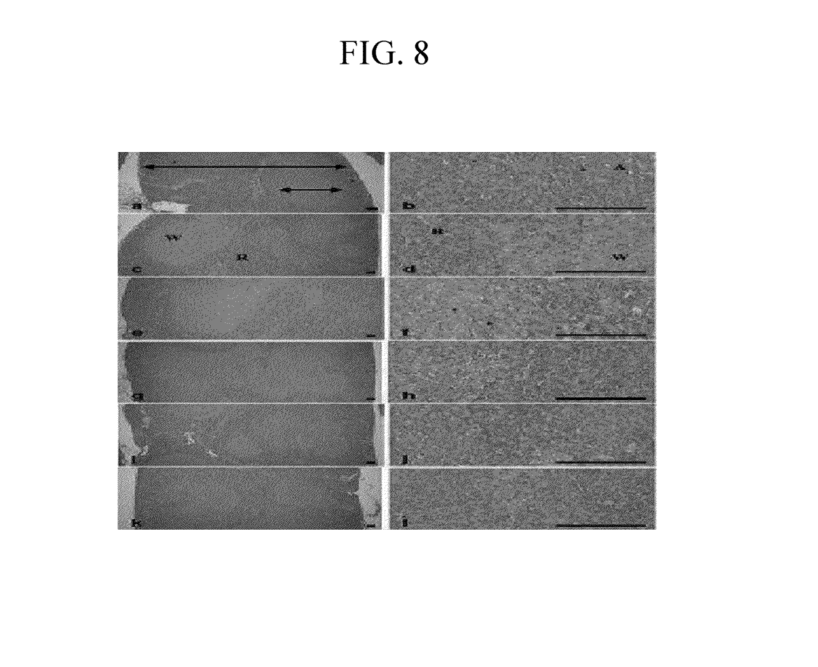

On the other hand, referring to FIG. 7 and FIG. 8, it is seen that the diameters of entire spleen and the white medulla were significantly (p<0.01) increased compared to those of the sham control group, and the number of white medulla was remarkably increased, in order words, the spleen was enlarged, and the lymphocyte was grown in the RA-induced control (RA control) group. The enlargement of spleen was observed to be remarkably suppressed by administering the three doses of Polycan, on the other hand, the group administered diclofenac sodium even significantly (p<0.01) increased the thickness of entire spleen compared to the RA control group.

It is estimated that the efficacy of the diclofenac sodium was caused by injection administration instead of oral administration, so it was a second increase caused by increasing the inflammation reactivity, but the Polycan efficacy was caused by the immune regulating effects.

From the results, it is seen that the groups administered Polycan at 21.25 mg/kg and 42.5 mg/kg had clear dose dependency, but the group administered 85 mg/kg had a similar or somewhat decreased efficacy to the group administered 42.5 mg/kg.

The entire thymus thickness of the RA control was changed by 2.84% compared to that of the sham control group, and the groups administered diclofenac sodium and Polycan at 21.25, 42.5, and 85 mg/kg changed by −2.80, 1.45, 6.17, and 4.85%, respectively, compared to the RA control group.

The thickness of the thymus cortex of the RA control was changed by 4.37% compared to that of the sham control group, and the groups administered diclofenac sodium and Polycan at 21.25, 42.5, and 85 mg/kg changed by 18.83, 4.37, 9.82, and 16.30%, respectively, compared to the RA control group. In addition, referring to the following Table 1, it is understood that the entire thymus thickness and the thickness of the cortex of the treated group were increased.

TABLE 1

| | Total | Cortex |
|---|---|---|
| | thickness | thickness |
| Group | (mm/lobules) | (mm/lobules) |
| Sham control | 1.941 ± 0.532 | 1.074 ± 0.273 |
| RA control | 1.997 ± 0.255 | 1.121 ± 0.194 |
| Diclofenac sodium | 1.941 ± 0.916 | 1.333 ± 0.705 |
| treated group | | |
| Polycan 21.25 mg/kg | 2.025 ± 0.354 | 1.170 ± 0.192 |
| Polycan 42.5 mg/kg | 2.120 ± 0.393 | 1.232 ± 0.201 |
| Polycan 85 mg/kg | 2.093 ± 0.403 | 1.305 ± 0.255 |

The entire spleen thickness of the RA control was changed by 24.32% compared to that of the sham control group, and the groups administered diclofenac sodium and Polycan at 21.2 5, 42.5 and 85 mg/kg changed by 17.49, −6.71, −11.12, and −10.44%, respectively, compared to the RA control group.

### Experimental Example 3-5

### Spleen White Medulla Analysis of Animal Model

The number of spleen white medulla of the RA control group was changed by 19.40% compared to that of the sham control group, and the group administered diclofenac sodium and Polycan at 21.25, 42.5, and 85 mg/kg changed by 5.00, −6.25, −5.00, and −13.75%, respectively, compared to the RA control group.

The diameter of the spleen white medulla of the RA control group was changed by 24.09% compared to that of the sham control group, and the groups administered diclofenac sodium and Polycan at 21.25, 42.5, and 85 mg/kg changed by −8.14, −16.99, −22.65, and −22.01%, respectively, compared to the RA control group. Additionally, referring to the following Table 2, it is understood that the entire thickness, the number of white medulla, and the thickness of the white medulla of the treated group were decreased.

In the following table, ±SD (average±standard deviation) of 8 animal models is shown and compared to that of the sham control group at *p<0.01 and **p<0.05. \p<0.01 compared to the rheumatoid arthritis control (RA control).

TABLE 2

| | | | Thickness of |
|---|---|---|---|
| | Entire thickness | Number of | white medulla |
| | (mm/central | white medulla | (mm/white |
| Group | part) | (/1 mm²) | medulla) |
| Sham control | 3.180 ± 0.218 | 8.375 ± 1.768 | 0.865 ± 0.118 |
| RA control | 3.954 ± 0.279^{*} | 10.000 ± 0.926 | 1.073 ± 0.119^{*} |
| Diclofenac sodium treated | 4.464 ± 0.58^{*†} | 10.500 ± 2.121 | 0.986 ± 0.304 |
| group | | | |
| Polycan 21.25 mg/kg | 3.689 ± 0.223^{*} | 9.375 ± 2.134 | 0.891 ± 0.076^{†} |
| Polycan 42.5 mg/kg | 3.515 ± 0.247^{**†} | 9.500 ± 1.604 | 0.830 ± 0.056^{†} |
| Polycan 85 mg/kg | 3.541 ± 0.281^{**†} | 8.625 ± 1.408 | 0.837 ± 0.067^{†} |

### Experimental Example 3-6

### Analysis of Proteoglycan Loss and Erosion

It is known that proteoglycan of articular cartilage histologically disappears in collagen-induced arthritis, and thus the erosion, which is typical in RA, is observed [Williams et al., 1992; Dudler et al., 2000; van Holten et al., 2004; Kim et al., 2007]. From the results of the present test, it is seen that proteoglycan of the femur and tibia articular cartilage of the knee joint of the RA-induced control group was significantly decreased (p<0.01) compared to that of the sham control group, the erosion score was increased, and the cartilage thickness was decreased.

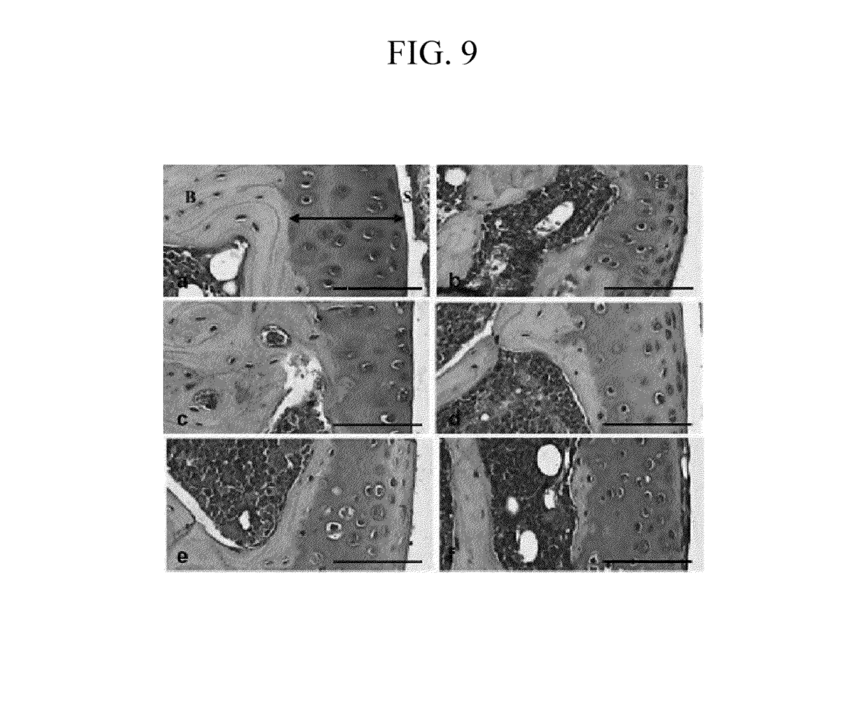

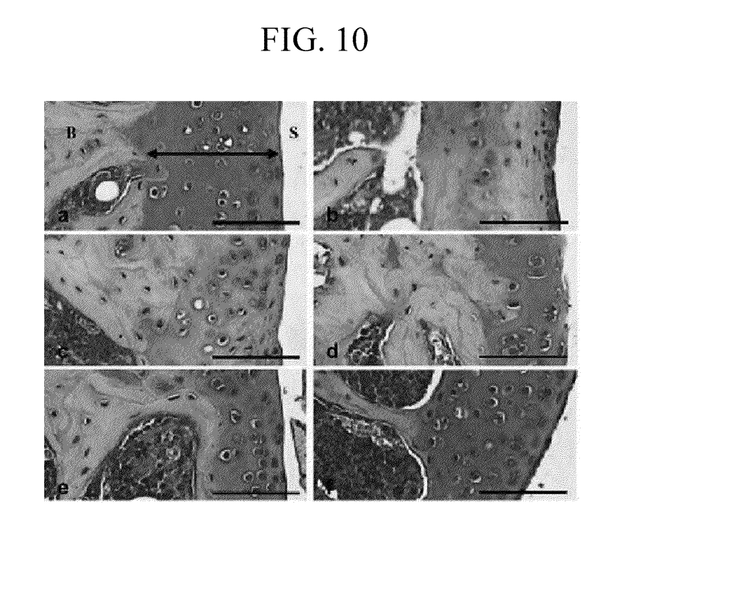

On the other hand, referring to FIG. 9 and FIG. 10, it was observed that collagen-induced RA opinion was remarkably suppressed in all groups administered Polycan except the 21.25 mg/kg administration group in the tibia. In the group administered 21.25 mg/kg of Polycan, it is seen that the proteoglycan loss was significantly suppressed (p<0.05), but it was observed that the erosion score and the thickness of tibia joint cartilage were similar to those of the RA control group.

On the other hand, the collagen-induced RA opinion was remarkably suppressed by administering diclofenac sodium. Diclofenac sodium is a non-steroidal anti-inflammation agent, and is well known for efficacy on collagen-induced RA by suppressing prostaglandin synthesis, and has been used as a comparative drug for the anti-RA efficacy evaluation of various materials [Sanchez-Pernaute et al., 1997; Rordorf et al., 2005].

### Experimental Example 3-7

### Anatomy of Femur Articular Cartilage of Animal Model

In the RA control group, the Safranin O score of femur articular cartilage was changed by 1000.00% compared to that of the sham control group, and the groups administered diclofenac sodium and Polycan at 21.25, 42.5, and 85 mg/kg changed by −63.64, −36.36, −63.64, and −59.09%, respectively, compared to the RA control group.

The erosion score of the femur articular cartilage of the RA control group was changed by 400.00% compared to that of the sham control, and the groups administered diclofenac sodium and Polycan at 21.25, 42.5, and 85 mg/kg changed by −36.00, −20.00, −44.00, and −44.00%, respectively, compared to the RA control group.

The thickness of the femur joint cartilage of the RA control group was changed by −43.79% compared to that of the sham control, and the groups administered diclofenac sodium and Polycan at 21.25, 42.5, and 85 mg/kg changed by 333.70, 21.12, 44.14 and 64.65%, respectively, compared to the RA control group.

Referring to the following Table 3, the treated groups had remarkably decreased Safranin level, erosion level, and thickness of the femur articular cartilage compared to the control group. In addition, the following table shows ±SD of 16 joints and a comparison with the sham control group at p<0.01 and **p<0.05. \p<0.01 compared with the rheumatoid arthritis control (RA control) group.

TABLE 3

| | | | Thickness of the |
|---|---|---|---|
| | Safranin level | Erosion level | femur articular |
| Group | (Max = 3) | (Max = 3) | cartilage (mm) |
| Sham control | 0.125 ± 0.342 | 0.313 ± 0.479 | 0.193 ± 0.030 |
| RA control | 1.375 ± 0.957^{*} | 1.563 ± 1.094^{*} | 0.108 ± 0.025^{*} |
| Diclofenac sodium | 0.500 ± 0.577 | 1.000 ± 0.000^{**} | 0.470 ± 0.674^{††} |
| treated group | | | |
| Polycan 21.25 mg/kg | 0.875 ± 0.71 9^{*} | 0.250 ± 0.683^{*} | 0.131 ± 0.030^{*††} |
| Polycan 42.5 mg/kg | 0.500 ± 0.516^{†} | 0.875 ± 0.500^{**} | 0.156 ± 0.024^{*††} |
| Polycan 85 mg/kg | 0.563 ± 0.629^{††} | 0.875 ± 0.691^{**} | 0.178 ± 0.046^{†} |

### Experimental Example 3-8

### Anatomy of Tibia Articular Cartilage of Animal Model

The Safranin O score of the tibia articular cartilage of the RA control group was changed by 209.09% compared to that of the sham control group, and the groups administered diclofenac sodium and Polycan at 21.25, 42.5 and 85 mg/kg changed by −29.41, −29.41, −50.00, and −73.53%, respectively, compared to the RA control group.

The erosion score of the tibia articular cartilage was changed in the RA control group by 966.67% compared to that of the sham control group. The groups administered diclofenac sodium and Polycan at 21.25, 42.5, and 85 mg/kg changed by −75.00, −6.25, −46.88, and −46.88%, respectively, compared to the RA control group.

The thickness of the tibia articular cartilage of the RA control group was changed by −43.00% compared to that of the normal control group, and the groups administered diclofenac sodium and Polycan at 21.25, 42.5, and 85 mg/kg changed by 38.75, 0.67, 14.08, and 36.64%, respectively, compared to the RA control group.

Referring to the following Table 4, it is understood that the Safranin level and erosion level of the treated groups tended to be decreased compared to those of the rheumatoid arthritis control (RA control), and the thickness of the tibia joint cartilage was increased. In addition, the following table shows results of ±SD of 16 joints and a comparison to the sham control at p<0.01 and **p<0.05. It was compared to the rheumatoid arthritis control (RA control) group at †p<0.01.

TABLE 4

| | Safranin | Erosion | Thickness (mm) |
|---|---|---|---|
| | level | level | of tibia articular |
| Group | (Max = 3) | (Max = 4) | cartilage |
| Sham control | 0.688 ± 0.704 | 0.188 ± 0.403 | 0.213 ± 0.038 |
| RA control | 2.125 ± 0.885^{*} | 2.000 ± 1.265 | 0.122 ± 0.050^{*} |
| Diclofenac sodium | 1.500 ± 0.577 | 0.500 ± 1.000 | 0.169 ± 0.024^{††} |
| treated group | | | |
| Polycan 21.25 mg/kg | 1.500 ± 0.966^{*††} | 1.875 ± 1.455 | 0.122 ± 0.038^{*} |
| Polycan 42.5 mg/kg | 1.063 ± 0.929^{†} | 1.063 ± 0.929 | 0.139 ± 0.043^{*} |
| Polycan 85 mg/kg | 0.563 ± 0.629^{†} | 1.063 ± 0.998 | 0.166 ± 0.043^{*†} |

### Experimental Example 4

### Toxicity Test of ‘β-1,3-1,6-branched D-glucan’ (Polycan™)

### Experimental Example 4-1

### Mouse Preparation

Twenty 6-week-old male ICR mice and 20 female ICR mice (Charles River, Japan) were each prepared. Groups of 5 mice were put into polycarbonate cages at a temperature of 20-25° C. and humidity of 30-35%. The day and night cycle was set in 12 hours:12 hours. In addition, water was supplied without limitation, and all mice were starved for the night before being sacrificed.

### Experimental Example 4-2

### Objects and Mixing

Polycan™ (Glucan Corp. Ltd., Korea) is a brown phlegmatic but uniform solution. The Polycan was stored in a refrigerator at 4° C. Each of male and female mice groups was orally administered the object material at 1000, 500, and 250 mg/kg. The mice were divided and numbered as in the following Table 5.

TABLE 5

| | | Number | | Total |
|---|---|---|---|---|
| | | of | Experimental | administration |
| Group | Sex | animals | animal number | amount (mg/kg) |
| GM | Male | 5 | G0B-01-G0M-05 | 0 |
| G1M | Male | 5 | G1B-01-G0M-05 | 1000 |
| G2M | Male | 5 | G2B-01-G0M-05 | 500 |
| G3M | Male | 5 | G3B-01-G0M-05 | 250 |
| G0F | Female | 5 | GOF-01-G0F-05 | 0 |
| G1F | Female | 5 | G1F-01-G1F-05 | 1000 |
| G2F | Female | 5 | G2F-01-G2F-05 | 500 |
| G3F | Female | 5 | G3F-01-G3F-05 | 250 |

### Experimental Example 4-3

### Statistic Analysis

LD₅₀ was calculated according to the Probit method. Statistic analysis was performed using SPSS (Release 6.1.3, SPSS Inc., USA) applied in Windows.

### Experimental Example 4-4

### Results

Each administration amount was changed according to the conditions shown in Table 5 and oral administered to the mice. The survival rate after each day is shown in the following Table 6.

TABLE 6

| Group ID | | Numbered of days | | | | | | | | | | | | | | Total |
|---|---|---|---|---|---|---|---|---|---|---|---|---|---|---|---|---|
| Male | G0M | 0 | 1 | 2 | 3 | 4 | 5 | 6 | 7 | 8 | 9 | 10 | 11 | 12 | 13 | 0/5 0% |
| | G1M | 0/5 | 0/5 | 0/5 | 0/5 | 0/5 | 0/5 | 0/5 | 0/5 | 0/5 | 0/5 | 0/5 | 0/5 | 0/5 | 0/5 | 0/5 0% |
| | G2M | 0/5 | 0/5 | 0/5 | 0/5 | 0/5 | 0/5 | 0/5 | 0/5 | 0/5 | 0/5 | 0/5 | 0/5 | 0/5 | 0/5 | 0/5 0% |
| | G3M | 0/5 | 0/5 | 0/5 | 0/5 | 0/5 | 0/5 | 0/5 | 0/5 | 0/5 | 0/5 | 0/5 | 0/5 | 0/5 | 0/5 | 0/5 0% |
| female | G0F | 0/5 | 0/5 | 0/5 | 0/5 | 0/5 | 0/5 | 0/5 | 0/5 | 0/5 | 0/5 | 0/5 | 0/5 | 0/5 | 0/5 | 0/5 0% |
| | G1F | 0/5 | 0/5 | 0/5 | 0/5 | 0/5 | 0/5 | 0/5 | 0/5 | 0/5 | 0/5 | 0/5 | 0/5 | 0/5 | 0/5 | 0/5 0% |
| | G2F | 0/5 | 0/5 | 0/5 | 0/5 | 0/5 | 0/5 | 0/5 | 0/5 | 0/5 | 0/5 | 0/5 | 0/5 | 0/5 | 0/5 | 0/5 0% |
| | G3F | 0/5 | 0/5 | 0/5 | 0/5 | 0/5 | 0/5 | 0/5 | 0/5 | 0/5 | 0/5 | 0/5 | 0/5 | 0/5 | 0/5 | 0/5 0% |

As shown in Table 6, it is understood all the female and male mice survived. Therefore, LD ₅₀ and approximate LD of both female and male mice after oral administration of Polycan were estimated to be over 1000 mg/kg.

While this invention has been described in connection with what is presently considered to be practical exemplary embodiments, it is to be understood that the invention is not limited to the disclosed embodiments, but, on the contrary, is intended to cover various modifications and equivalent arrangements included within the spirit and scope of the appended claims.

1. A composition for treating, preventing, or alleviating arthritis comprising a β-1,3-1,6-branched D-glucan.

2. The composition of claim 1, wherein the β-1,3-1,6-branched D-glucan comprises a repeating unit represented by following Formula I: 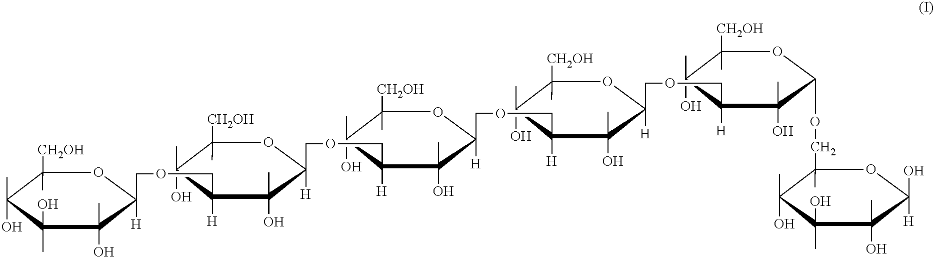
wherein an organic acid is bound with the branched glucose that is linked via a linkage between 1 and 6 positions of two glucose molecules.

3. The compound of claim 1, wherein the β-1,3-1,6-branched D-glucan is characterized in that glucoses are linked via beta-1,3 linkage between two adjacent glucose molecules to form a linear chain, and a glucose is branched from the chain at every 1 to 20 of the glucoses in the chain, wherein the branched glucose is linked to the chain via a linkage between 1 and 6 positions of two glucose molecules, and wherein the branched glucose is bound with an organic acid.

4. The compound of claim 2, wherein the organic acid is selected from the group consisting of lactic acid, oxalic acid, oxalacetic acid, fumaric acid, malic acid, succinic acid, acetic acid, butyric acid, palmitic acid, tartaric acid, ascorbic acid, uric acid, sulfonic acid, sulfinic acid, phenol, tartaric acid, formic acid, citric acid, isocitric acid, alpha-ketoglutaric acid, succinic acid, hexane, PGAL, DPGA, and PGA.

5. The compound of claim 4, wherein the organic acid is lactic acid.

6. The compound of claim 1, wherein the arthritis is osteoarthritis or rheumatoid arthritis.

7-9. (canceled)

10. A method of treating, preventing, or alleviating arthritis comprising administering a pharmaceutically effective amount of β-1,3-1,6-branched D-glucan.

11. The method of claim 10, wherein the arthritis is osteoarthritis or rheumatoid arthritis.

12. The method of claim 10, comprising administering 21.25 mg/kg to 85 mg/kg of the β-1,3-1,6-branched D-glucan.

13. The compound of claim 3, wherein the organic acid is selected from the group consisting of lactic acid, oxalic acid, oxalacetic acid, fumaric acid, malic acid, succinic acid, acetic acid, butyric acid, palmitic acid, tartaric acid, ascorbic acid, uric acid, sulfonic acid, sulfinic acid, phenol, tartaric acid, formic acid, citric acid, isocitric acid, alpha-ketoglutaric acid, succinic acid, hexane, PGAL, DPGA, and PGA.

14. The compound of claim 13, wherein the organic acid is lactic acid.

15. The method of claim 10, wherein the β-1,3-1,6-branched D-glucan comprises a repeating unit represented by following Formula I: 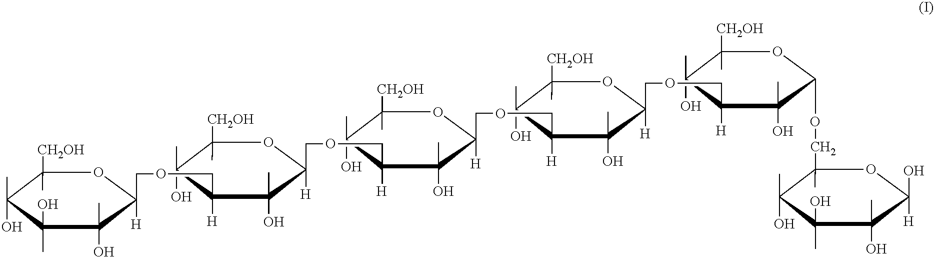
wherein an organic acid is bound with the branched glucose that is linked via a linkage between 1 and 6 positions of two glucose molecules.

16. The method of claim 10, wherein the β-1,3-1,6-branched D-glucan is characterized in that glucoses are linked via beta-1,3 linkage between two adjacent glucose molecules to form a linear chain, and a glucose is branched from the chain at every 1 to 20 of the glucoses in the chain, wherein the branched glucose is linked to the chain via a linkage between 1 and 6 positions of two glucose molecules, and wherein the branched glucose is bound with an organic acid.

17. The method of claim 15, wherein the organic acid is selected from the group consisting of lactic acid, oxalic acid, oxalacetic acid, fumaric acid, malic acid, succinic acid, acetic acid, butyric acid, palmitic acid, tartaric acid, ascorbic acid, uric acid, sulfonic acid, sulfinic acid, phenol, tartaric acid, formic acid, citric acid, isocitric acid, alpha-ketoglutaric acid, succinic acid, hexane, PGAL, DPGA, and PGA.

18. The compound of claim 16, wherein the organic acid is selected from the group consisting of lactic acid, oxalic acid, oxalacetic acid, fumaric acid, malic acid, succinic acid, acetic acid, butyric acid, palmitic acid, tartaric acid, ascorbic acid, uric acid, sulfonic acid, sulfinic acid, phenol, tartaric acid, formic acid, citric acid, isocitric acid, alpha-ketoglutaric acid, succinic acid, hexane, PGAL, DPGA, and PGA.

19. The method of claim 17, wherein the organic acid is lactic acid.

20. The method of claim 18, wherein the organic acid is lactic acid.

21. The method of claim 10, wherein the pharmaceutically effective amount of a β-1,3-1,6-branched D-glucan is administered orally .

22. The method of claim 10, wherein the pharmaceutically effective amount of a β-1,3-1,6-branched D-glucan is administered via parenteral route.

23. The method of claim 10, wherein the pharmaceutically effective amount of a β-1,3-1,6-branched D-glucan is administered via injection.
